(19) United States
(12) Patent Application Publication
(10) Pub. No.: US 2024/0328933 A1
(43) Pub. Date: Oct. 3, 2024

(54) METHOD AND APPARATUS FOR USE IN OPTICAL GAS ABSORPTION MEASUREMENTS

(71) Applicant: Servomex Group Limited, Crowborough (GB)

(72) Inventors: Bahram Alizadeh, Crowborough (GB); Martin Lopez, Crowborough (GB); Michael Lawson, Crowborough (GB); Michael Ratcliffe, Crowborough (GB)

(73) Assignee: Servomex Group Limited, Crowborough (GB)

(21) Appl. No.: 18/619,846

(22) Filed: Mar. 28, 2024

(30) Foreign Application Priority Data
Mar. 31, 2023 (GB) 2304895.2
Dec. 29, 2023 (GB) 2320141.1
Mar. 27, 2024 (GB) 2404427.3

(51) Int. Cl. G01N 21/31; G01N 33/00

(52) U.S. Cl. CPC G01N 21/31; G01N 33/0027; G01N 2201/06113

(57) ABSTRACT

An apparatus for use in absorption spectroscopy, comprising: at least one source of electromagnetic radiation for transmitting electromagnetic radiation along an optical path that passes through a gas measurement volume, towards at least one detector; at least one detector to detect the transmitted electromagnetic radiation after passing through the gas measurement volume and to provide an output signal indicative of the detected electromagnetic radiation; and an analyser connected to the at least one detector to receive the output signal and analyse the effects of absorption by at least one gas species within the gas measurement volume for at least one wavelength range of the transmitted electromagnetic radiation, thereby to detect or measure a parameter of the at least one gas species; wherein at least one source or detector comprises a Chip-on-Board (COB) component comprising a solid-state source and/or detector of electromagnetic radiation mounted onto a substrate in a COB configuration.

### CROSS REFERENCE TO RELATED APPLICATIONS

This patent application is entitled to claim priority from UK Patent Application No. 2404427.3 filed on Mar. 27, 2024, UK Patent Application No. 2320141.1 filed on Dec. 29, 2023, and UK Patent Application No. 2304895.2 filed on Mar. 31, 2023, which are incorporated herein by reference.

### FIELD OF THE INVENTION

This invention relates to apparatus and methods for use in gas absorption spectroscopy in general and in tuneable diode laser absorption spectroscopy (TDLS) and non-dispersive infrared spectroscopy in particular. The invention has applications in the detection and measurement of one or more species in a gas, such as those produced by an artificial or natural process such as an industrial, medical or physiological process.

### BACKGROUND OF THE INVENTION

An example absorption measurement system consists of a source of electromagnetic radiation such as a tuneable laser source, for example a tuneable diode laser (TDL), or a broadband source such as an incandescent source or light emitting diode (LED) in combination with a wavelength range selective element, such as an optical passband filter or grating. The source emits a beam of electromagnetic radiation that is focussed on to a detector, which may be a solid-state photovoltaic, photoconductive, photomultiplier, pyrometer, thermopile or bolometer detector. The substance that is to be analysed is positioned between the electromagnetic radiation source and the detector, so that the electromagnetic radiation incident on the detector may be modified (i.e. reduced by absorption) by its path through the substance. The modifications to the electromagnetic radiation enable various parameters of the measurand gas or other substance to be determined by a signal processing system that is coupled to the detector. Such measurements may take place across an in-situ gas sample measurement volume, such as a process stack or within a measurement gas sample cell. FIG. 1 illustrates a basic extractive absorption measurement apparatus, where 101 is the source of electromagnetic radiation, 102 is the sample cell with transmissive windows at each end and gas inlet 103 and outlet 104, and the detector 105. A signal processing system 106 is used to process the detector signal and produce the measurand concentration based on a calibrated algorithm. In some cases, the substance to be analysed is a gas produced by an industrial, medical or natural process and the measurand may be a parameter of one or more chemical species that are present in this gas. References to a ‘measurand gas’ or ‘measurand species’ in this patent specification are intended to refer to a gas or gas species for which one or more parameters are to be measured or detected; the ‘measurand’ is the presence of a gas species or a measurable parameter of the gas species. Examples of measurand species include, but are not limited to, gaseous water, O₂, NO, NO₂, CO, CO₂ and hydrocarbons such as methane or ethane. These measurements are often used for optimising process efficiency and for the monitoring and/or minimising of the production of pollutants and greenhouse gases, as well as for the monitoring and/or optimisation of physiological well-being. The presence and/or amount fraction (concentration) of one or more of these measurand species may be determined by absorption spectroscopy measurements using one or more TDLs. It is known to convert observed changes in electromagnetic radiation intensities to useful physical parameters, such as concentrations and temperatures, but this typically requires a series of assumptions to be made regarding the measurand species and measurement apparatus. The term electromagnetic radiation covers a very broad wavelength range and often, but not exclusively, absorption spectroscopy measurements occur in the ultraviolet, visible and infrared regions of the electromagnetic spectrum. Illustrations within this patent specification will be given for the infrared light region of the electromagnetic spectrum, for obtaining absorption spectroscopy measurements corresponding to certain molecular vibrational energy transitions, but the same principles may be applied to other relevant wavelength regions and should not be considered limited to this spectral region. For ease of expression, throughout this patent specification, the terms electromagnetic radiation or light may be used interchangeably and should be interpreted as being equally applicable to the ultraviolet, visible, infrared or other relevant range of the electromagnetic spectrum unless stated otherwise.

In operation of an example laser gas analyser system, the wavelength of the beam emitted by a TDL is scanned over a range of wavelengths including one or more absorption lines of the measurand gas species. At specific wavelengths within the range of wavelengths scanned, light is absorbed by the measurand gas, and these spectral absorption lines can be detected by measuring changes in the light flux through the substance to be analysed.

In operation of an example broadband gas analyser system, the source emits over a relatively broad wavelength range (encompassing at least one relevant absorption line), even if limited via the use of an optical passband filter or diffraction grating, and the throughput intensity is modified by absorption simultaneously occurring over one or more absorption lines of the measurand gas species. The integrated effect of the spectral absorption line(s) can be detected by measuring changes in the light flux through the substance to be analysed due to the presence of the measurand.

Absorption lines have characteristic “shapes” in wavelength space that are dependent on the intrinsic physical properties of the gas species (e.g. bond angles, bond lengths, number of electrons), as well as the extrinsic physical properties (e.g. velocity, temperature) and properties of the environment (e.g. pressure, composition of surroundings, etc.). The following paragraphs attempt to give a brief overview of the mechanisms by which these shapes are produced and to give some insight about current practical limitations concerning the recovery of useful properties, such as concentration and temperature, where other perturbing factors are not, or cannot be, well-defined.

In the context above, an absorption “line” is an observable change in light transmission, which coincides with a frequency (wavelength) interval over which a photon may induce a gas molecule to transit from one quantum state to another. The probability that a photon with a particular wavelength and polarisation will elicit transition of quantum states is given by the transition's absorption cross section. The transition dependent cross section can be estimated from first principles, but, in practice, it is typically measured experimentally with high accuracy.

Since the interaction of light with matter is inherently quantum in nature, the degree to which a measurand gas species with a fixed chemical identity absorbs light of a given frequency is determined not only by its number density in the measurement path but also by its precise quantum mechanical state. For any given gas species, there will exist numerous quantum states with spectroscopically distinct absorption properties.

From the Heisenberg uncertainty principle, it follows that the energies of quantum states cannot be precisely defined. This uncertainty causes a smearing of the photon energies required to elicit a transition between two states, preventing spectral lines from becoming infinitely narrow. The statistical impact of different broadening mechanisms can be gathered into a frequency dependent term referred to as the “spectral line-shape”.

As mentioned earlier, the energies of a molecule's quantum states can be perturbed by its physical environment. For example, outside of a perfect vacuum, molecules collide with other particles. These collisions, like photons, can induce changes in quantum state of the molecule, such that the natural lifetime of the original states is shortened. In gaseous states, the influence of this collisional broadening, to a great extent, resembles natural broadening and, accordingly, its influence is generally treated as a modification to the Lorentzian full width half maximum (FWHM) height. However, in contrast to natural line broadening, the pressure contribution has a more complex relationship with the absorption line shape. Pressure broadening, which is applicable to gases only, for a given set of conditions, can be deduced by physically measuring the line shape and carefully subtracting other known broadening mechanisms. This is often impractical and frequently the collisional broadening is only catalogued at “Normal Temperature and Pressure” (NTP) in the absence of other gases and in air. The resultant values may then be fed into mathematical models, so that linewidths corresponding to different conditions may be extrapolated.

The thermal motion of a measurand gas will cause its constituent molecules to possess a range of velocities in relation to the light source. If one considers these molecules to be “observers” of incoming photons, these photons will appear red or blue shifted. At any given temperature, to conserve energy and momentum, lighter molecules will travel faster, on average, compared to those which are more massive. Therefore, thermal broadening has a positive relationship to temperature and an inverse relationship to molecular mass.

From the paragraphs above, it follows that for most practical spectroscopic applications, where temperature and pressure have an appreciable effect, the resultant transition profile is neither completely Lorentzian nor Gaussian in shape. For this reason, it is known to make use of a “Voigt” profile, which is a convolution of the Lorentzian and Gaussian line shapes.

It is possible to accurately determine, for example, changes of the volume density of a measurand gas in a straightforward fashion, provided the perturbing factors listed above are stable and there are no variations in incident light intensity and measurement path length. Such variations may occur, for example, during thermal flexing and mechanical vibrations of the measurement chamber. Variations in incident light intensity may be caused by a number of factors other than absorbing molecular density changes. For example, variations can be caused by intrinsic fluctuations in the laser output, changes in ambient light intensity levels and/or obscuration in the process sample stream, which may be caused by any combination of dust, tar, corrosion or optical beam misalignment. In an extractive or laboratory setting such environmental factors may be limited, for example, by chemical and/or physical filtration to eliminate dust and/or other physical contamination and the presence of undesired chemical species such as water, or mitigated, for example, by compensating for or controlling the ambient temperature and/or pressure. Even in this situation, compensation in light intensity fluctuations may still be desirable due to source instability or drift (as described, for example, in U.S. Pat. No. 9,546,902). The system described in the present patent specification is particularly well-suited for an in-line, extractive environment such as in a laboratory or equivalent environment, although it could also be employed in an in-situ measurement, such as a pipe or stack.

A further cause of measurement uncertainty can be present due to spatial averaging. This type of uncertainty arises due to the fact that the spectroscopic measurements described above reduce the state of a 3-dimensional system to a 1-dimensional transmittance value. This means that any variations over the pathlength will be averaged, so that all spatial information is completely lost. Assuming that these parameters are not constant along the measurement pathlength, which is especially the case for in-situ gas measurements, this can present major challenges for useful signal recovery.

Direct and indirect cross interference may also occur. This interference results from the effect that the partial pressure of a foreign gas has on the measurement, either through “direct” overlap of absorption lines within the measured wavelength range or from the “indirect” effect of the foreign gas colliding with the measurand gas and modifying the measurand gas absorption line. The composition of the measurement stream is seldom known with precision; if it were, the requirement for the measurement would be nullified and therefore it follows that the line shape produced by a fixed quantity of measurand gas may vary unpredictably if the partial pressures of foreign, broadening gases are not known. Direct cross interference, when caused by a foreign gas, will always result in some degree of additional indirect cross-interference. Depending on the spectroscopic technique used, this may have a significant impact on the measured transition intensity. For example, where wavelength modulation spectroscopy (WMS) is employed, the recovered line intensity is dependent on the ratio of applied amplitude modulation and transition linewidth. It follows that indirect cross interference, in this situation, necessitates a measurement technique whereby the line shape is constantly measured and used to normalise fluctuations in the transition intensity. Any uncertainty in the line shape arising from, for example, electronic noise, will be coupled into the measurement signal, to the detriment of its precision and accuracy. Alternatively, a measurement from a secondary sensor may be used as a normative input although, once again, uncertainties still couple into the measurement and multiple sensors may be needed to achieve full coverage of interferents.

Another potential cause of fluctuations in the optical detector signal, which is not due to direct fluctuations in the ambient light or laser output signal, is the occurrence of constructive and destructive optical interference (etalons) causing an oscillation in the detector signal as the laser is scanned across the measurement wavelength range. Optical interference will even be present, to some degree, with an incoherent broadband light source, due to random effects, but the use of coherent laser light, for example, means that any reflections at any optical surfaces or interfaces along the optical path from the laser output to the detector surface (for example from surfaces/interfaces such as, but not limited to, windows, lenses and reflective interfaces), lead to the production of reflected light with a phase difference in comparison with the incident light, hence leading to optical interference, where the light rays interact. The phase relationship between this reflected light and the incident light may change with time due to such factors as temperature, vibration and pressure fluctuations, since these factors may cause changes in physical dimensions, density or refractive index.

The detector is integrating this optical interference when producing an intensity signal. Since the phase difference will vary with wavelength along the measure path, the symptoms of this optical interference (or etalons) are typically the production of oscillations on the signal baseline as the laser output is scanned across the wavelength measurement range. These combine with other distortions and cause measurement inaccuracies. The signal “baseline” is the signal that would be seen even if no absorbing signal were present, in other words, the “zero absorption” signal. This baseline signal is superimposed on the actual absorption signal when present. In an ideal world, the baseline would be a straight line (flat line centred at zero in perfect circumstances), but in practice this is never achieved. The baseline may not be perfectly flat across the scan range and may have fluctuations and other distortions (or “noise”), which may be of a random or systematic nature and include the above-mentioned oscillations. These oscillations may also be referred to as “fringe” signals in the case of optical interference. These various distortion effects, of whatever origin, lead to increased uncertainty in the determination of the absorption signal or signals, and hence increased uncertainty in the derived molecular density or concentration of the measurand gas. The uncertainty is compounded where the presence of indirect cross-interference necessitates the measurement of line shape in addition to transition intensity, since the periodic etalon fringes may cause the recovered signal to broaden or narrow.

Methods to decrease such optical interference include reducing reflective or partially reflective surfaces in the light path from source to detector that may form etalons, such as by minimising the number of optical components, using wedge windows rather than parallel face windows or using anti-reflection coating optimised for the desired wavelength range. However, it is impossible, in practice, to eliminate this interference effect by reducing reflective surfaces. Where a multi-pass cell is used, interference is unavoidable, as the beam path within a multi-pass cell will always create some amount of optical interference, which is usually significant when the cell is being used for trace level measurements.

Another method to reduce the impact of optical interference on the baseline is to measure and record a reference baseline when no measurand gas is present. This reference baseline may then be subtracted from the live signal to produce a cleaner signal to process. Whilst this may give an immediate improvement to the measurand determination uncertainty, it does not address oscillations on the baseline under changes in ambient conditions (particularly temperature) and hence the effectiveness of this technique is limited.

Another method involves the use of a piezo electric element or similar means to oscillate an active optical element such as a lens or mirror in the optical path. This has the effect of continuously varying the optical pathlengths and hence the phase variations and resultant optical interference. This results in blurring or smoothing down the periodic oscillation on the baseline, through integration over time of the interference fringes formed and therefore reducing the overall effect. However, it adds complexity and cost and suffers from several problems due to using a moving element, such as reduced component lifetime and mechanical failure and, in practice, does not eliminate the problem completely. Moreover, most piezo electric elements require a sufficiently high voltage supply that makes operation in flammable hazardous areas unsuitable.

Other methods make use of the distinct transition line shapes, described earlier, and their difference from the periodic intensity fluctuations arising from optical self-interference. This might be carried out, for example, by least squares fitting a recovered spectrum to a suitable basis-vector which would resemble the expected spectrum in the absence of interference. This method has the advantage that further basis vectors can be employed that resemble the individual spectra of other cross-interfering gases in the measurement. Alternatively, a similar procedure may be carried in the frequency domain using convolution with a suitable kernel function, such as described in U.S. Pat. No. 10,234,378. These methods, however, are limited in that they require assumptions to be made regarding the transition line shape. If it deviates from the expected value, the resulting measurements become inaccurate. For example, if a direct-cross interferent is present in the measurement stream which is not accounted for in the corrective basis-set or frequency domain kernel, such approaches can be detrimental to measurement accuracy. Furthermore, if factors affecting the production of optical noise differ significantly from expected values, such as the free spectral range of the resultant etalon fringes, the efficacy of such approaches is reduced.

A recent novel method (described in UK Patent Application No. GB2113699.9) involves the application of electrical and/or magnetic fields to modify the absorption line characteristics due to the Zeeman and/or Stark effects, whilst leaving the optical interference effects unchanged. In this way, the baseline effect of the optical interference may be deduced and/or decreased or eliminated.

Under constant ambient conditions of pressure, temperature and background gas composition, the fractional strength of the electromagnetic absorption by a gas is dependent on the gas concentration, the fundamental properties of the gas (the extinction coefficient, which is dependent on the wavelength) and the pathlength through the absorbing gas. The mathematical relationship between these properties is described by the Beer Lambert Law. If a low gas concentration is desired to be determined, sensitivity for a particular gas may be enhanced by choosing a strong absorption line and/or a long pathlength. However, if the absorption of light is too strong, such as if a high measurand gas concentration is present and/or a long pathlength, non-linearities and/or absorption saturation may occur, reducing sensitivity with concentration change. Hence, the required sensitivity (which will determine the chosen wavelength and pathlength) is dependent on the required measurement range. The wavelength chosen for the excitation also has pragmatic considerations, such as the availability and cost of excitation sources and detectors. For example, longer wavelength infrared lasers (such as interband or quantum cascade lasers), which may have stronger absorption, may cost considerably more than near infrared lasers (such as vertical cavity surface emitting laser (VCSEL) or distributed feedback (DFB)), which operate using weaker overtone absorption bands. Also, for broadband measurements, infrared LEDs typically have increased cost and decreased output intensity, when longer wavelengths are chosen. Once the excitation wavelength or wavelength range has been selected, then the pathlength will be determined by the required measurement range. Long pathlengths may be achieved by simply using a long sample cell, but this may be (physically) impractical and introduce a slow time response due to the large cell volume, especially if the required pathlength is of several metres or more. Many of these issues may be mitigated, such as by using a multi-pass or folded path cell, such as a Herriott cell (FIG. 2(a)), White cell (FIG. 2(b)) or tuned optical cavity, which may reduce the overall cell size considerably. In FIG. 2(a), the source of electromagnetic radiation 201 is collimated by a lens 202 and directed into the Herriot cell folded optical geometry 203. Within this cell there are two concave mirrors 204 to steer the beam in multiple folds to increase the pathlength. The detector 205 measures the transmitted radiation intensity. Gas exchange ports 206 allow the sample or calibration gas mixtures to be passed in and out of the cell. In FIG. 2(b) the source of electromagnetic radiation 207 is collimated by a lens 208 and directed into the White cell optical geometry 209. Within this cell a set of planar mirrors 210 are used to steer the beam in multiple folds to increase the optical pathlength. The detector 211 measures the transmitted radiation intensity. Gas exchange ports 212 allow the sample or calibration gas mixtures to be passed in and out of the cell. In any of the above cases, optical treatment and/or focussing of the excitation beam will normally be required for optimal functionality, needing bespoke optical design elements, such as refractive and/or reflective elements, and precision alignment tuning is needed as part of the manufacturing process. High performance, long pathlength multi-pass or folded path cells will require the use of low temperature coefficient of expansion materials and close temperature control for best performance. In addition, multi-pass cells and optical cavities may be highly sensitive to vibration and contamination.

There are also instances where an intermediate length folded absorption pathlength may be desirable to obtain some extra absorption benefit in a more compact format, whilst not requiring the optical complexity and environmental sensitivity of a very long pathlength multi-pass cell. Novel devices addressing these issues are described in UK Patent Application No. 2304895.2. The devices disclosed in that patent application do not require beam shaping, or the combination of focusing optics with an individual optical tuning process (alignment or beam shaping), which are required by some absorption spectroscopy systems. The present invention, which is described below, may be implemented in such devices.

In some optical absorption spectroscopy systems, although the majority of the optical path between a source and a detector is within a measurement volume such as a sample cell, there is also a small part of the optical path between the radiation source and cell input optical element, and between the cell output optical element and the detector. In this patent specification, we will refer to these parts of the absorption spectroscopy system that are outside the detection/measurement volume but within the optical path as “dead volumes” or “dead zones” as the gas to be tested is not intentionally flowed into these zones. These will typically be zones that are unswept by any purge gas. If any target gas or optically interfering species is present in one of these dead volumes or zones that are within the optical path, this will have an additional absorption effect to the gas in the sample cell. This is illustrated in the earlier figures as 107 and 213. This absorption effect will be affected by environmental conditions, especially temperature.

Where solid-state lasers (e.g. TDLs) and/or LEDs are used as sources of electromagnetic radiation and photodiode detectors are used as sensors of electromagnetic radiation, these are normally supplied as locally encapsulated devices, typically in hermitically sealed, evacuated or inert gas-filled metal cans with a transmissive window, or as resin/adhesive encapsulated devices, any of which may be individually positioned or mounted directly onto a printed circuit board (PCB). The canned devices may also feature integrated thermo-electric coolers (TECs) and temperature sensors.

This may lead to one or more of the following disadvantages:
a relatively complicated and costly package format,
a relatively large footprint and vertical height, which has implications for spectrometer optic-mechanics,
a relatively high thermal capacity (thermal mass) for temperature control by a thermo-electric cooler (TEC),
a relatively high electrical capacity due to the capacitances formed between the individual build components—this can affect the frequency performance of the device,
a relatively high thermal loss route via thermal conductivity of the package electrodes,
an extra optical interface (e.g. a window or lens), causing:
   transmissive losses, and
   back reflections causing extra issues for TDLs of:
      feedback interference into the TDL, and
      formation of an etalon-creating cavity within the can (which may persist even when mitigated by the use of angled windows and/or anti-reflective coatings).

There is therefore a need to mitigate these issues relating to the use of electromagnetic radiation sources and/or photodetectors in gas absorption spectroscopy apparatus.

### SUMMARY

This patent specification describes exemplary methods and apparatus for spectroscopic absorption measurements, as set out in the accompanying claims, whereby at least one Chip-on-Board (COB) component is used for the source and/or detector of electromagnetic radiation.

A first aspect of the present invention provides an apparatus for use in absorption spectroscopy, comprising:
at least one source of electromagnetic radiation for transmitting electromagnetic radiation along an optical path that passes through a gas measurement volume, towards at least one detector;
at least one detector to detect the transmitted electromagnetic radiation after passing through the gas measurement volume and to provide an output signal indicative of the detected electromagnetic radiation; and
an analyser connected to the at least one detector to receive the output signal and analyse the effects of absorption by at least one gas species within the gas measurement volume for at least one wavelength range of the transmitted electromagnetic radiation, thereby to detect or measure a parameter of the at least one gas species;
wherein at least one source or detector comprises a Chip-on-Board (COB) component comprising a solid-state source and/or detector of electromagnetic radiation mounted onto a substrate in a COB configuration.

In another aspect, the invention provides a method of constructing an apparatus for use in absorption spectroscopy, comprising the steps of:
providing at least one source of electromagnetic radiation, for transmitting electromagnetic radiation along an optical path that passes through a gas measurement volume, towards at least one detector; and
providing at least one detector to detect the transmitted electromagnetic radiation after passing through the gas measurement volume and to provide an output signal indicative of the detected electromagnetic radiation;
wherein at least one source or detector comprises a solid-state source and/or detector of electromagnetic radiation, and the method further comprises:
mounting the solid-state source and/or detector onto a substrate in a Chip-on-Board (COB) configuration to form a COB component;
wire-bonding the COB component to form electrical connections between the COB component and connection pads provided on the substrate; and
encapsulating the COB component with a layer of protective material.

Advantageous additional features are set out in the appended claims, and the advantages of these features will be apparent from the following description.

Although the following sections will describe the advantages and implementation of COB for a laser diode source, other embodiments may use at least one of a COB diode laser, LED source, photodiode and/or any other appropriate solid-state source or detector. The use of a bare laser device in its native diverging cone of light chip format may be advantageous in some embodiments. There are several advantages to using the Chip-on-Board (COB) format for the laser device, such as: reduced cost; less packaging material; lower etalons; higher optical transmission; significantly reduced heating and/or cooling power needed to maintain the laser chip at an accurate setpoint temperature; and tighter temperature control, due to the lower thermal mass and higher thermal transfer efficiency. References to Chip-on-Board (COB) technology may describe the mounting of the bare component (e.g. VCSEL or other laser chip) in direct contact with the substrate, or with the (typically copper) plane of a PCB (see FIG. 9, described in more detail below).

### BRIEF DESCRIPTION OF DRAWINGS

Example systems and methods are described below with reference to the accompanying figures in which:

FIG. 1 shows a prior art arrangement of a spectroscopic absorption gas analysis system for making extractive sample gas measurements;

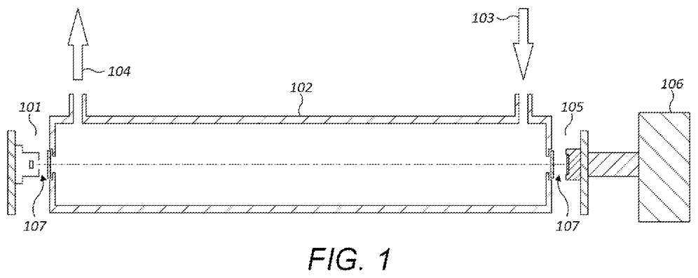

FIGS. 2(a) and 2(b) show prior art arrangements of a spectroscopic gas analysis system with a folded pathlength using multiple reflections, from a collimated light beam;

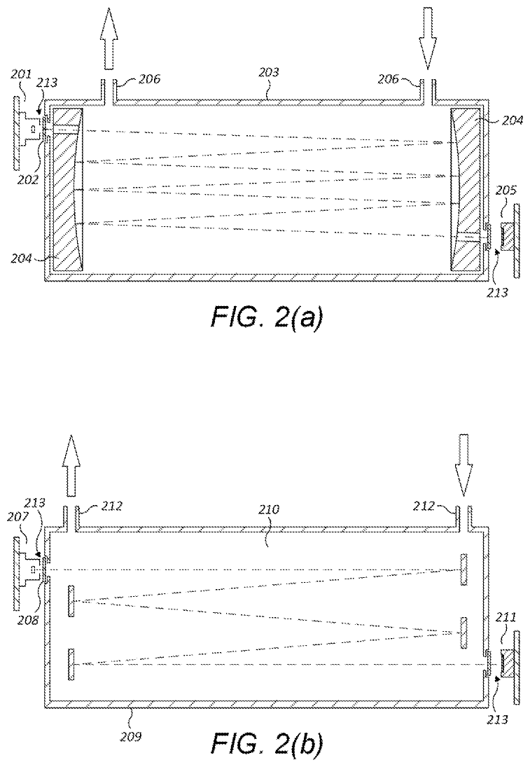

FIG. 3 shows an example spectroscopic gas analysis system;

FIG. 4 shows an example of a spectroscopic gas analysis system with structures similar to FIG. 3, where a combination of two or more folded path cells are used to increase the overall absorption pathlength;

FIG. 5 illustrates another example gas analysis apparatus implementing an alternative optical geometry embodiment with longer pathlength per cell length;

FIG. 6 shows the inclusion of a wavelength lock cell into a device;

FIG. 7 shows examples of a spectroscopic gas analysis system, where an electric and/or magnetic field is applied to one or more regions;

FIGS. 8(a) and 8(b) show examples of spectroscopic gas analysis systems, where sample gas, a purge gas or a reference gas is flowed through the dead space, or a scrubber is applied to the source and/or detector dead space regions;

FIG. 8(c) shows an example spectroscopic gas analysis system with a transmissive material filling a dead space adjacent a light source and a detector;

FIGS. 9(a), 9(b), 9(c) and 9(d) illustrate a Chip-on-Board design of a laser source for use in a spectroscopic gas analysis system, with an optional transmissive filler material shown in each of FIGS. 9(b), 9(c) and 9(d);

FIGS. 10(a) to 10(d) show examples of thermal coupling of source and/or detector components for use in a spectroscopic gas analysis system, in a range of illustrative mounting formats, using a COB design; and

FIG. 11(a) illustrates the effect of residual oxygen in a void within the optical path of transmitted radiation, and FIG. 11(b) illustrates the same apparatus with a silicone-based gel introduced into the void to displace the residual oxygen.

### DETAILED DESCRIPTION

This patent specification describes exemplary arrangements for a spectroscopic absorption measurement apparatus, whereby at least one Chip-on-Board (COB) component is used for the source and/or detector of electromagnetic radiation. Chip-on-Board (COB) tuneable laser diodes (TDLs) used as the radiative source may typically be VCSELs (vertical cavity surface-emitting lasers), DFBs (distributed feedback lasers) or DMs (discrete mode lasers).

Various materials may be used for solid-state laser sources, such as InP, GaAs, InGaAs, InAlGaAs and InAsSb in the infrared and, similarly, corresponding photodiodes in the infrared may be based on silicon, InSb, InGaAs, InAsP, InAlGaAs, InAsSb, PbS or PbSe, or may use other suitable material systems. For certain preferred embodiments, VCSELs are especially suited to the COB format for absorption spectrometers, since they emit the light vertically and have a mainly circular profile cross section, and hence are optimally employed for compact optical and thermal integration.

However, other embodiments may best suit mounting of components at an angle relative to the substrate. In certain embodiments, DFBs and DMs may require to be mounted at an angle to direct their electromagnetic radiation output and hence may have decreased thermal and physical integrative compactness compared to VCSELs and may be provided with a bespoke sub-mount for optimal thermal and opto-mechanical arrangement. In particular, the sub-mount may be configured to provide improved thermal coupling between a radiation source component and the substrate, when the required angle between the component and the substrate is such that it is not possible to mount the component flat on the substrate. In such arrangements, the optimal design of the sub-mount may be determined theoretically and/or empirically for efficient optical throughput into the gas cell with low back scatter/reflection. In a similar manner, a solid-state detector, such as a photodiode, may be mounted directly as a COB component or may be provided with a sub-mount to minimise optical back reflections. The angle at which a detector is mounted with respect to the substrate by means of the sub-mount may be selected based on the geometry of the detector position relative to the sample cell. The sub-mount may be wire-bonded to the PCB.

It should be noted that when a component, such as a laser chip, is mounted directly on a PCB, it can be difficult to verify the characteristics of the component before completion of the PCB assembly. Where a component is attached to a sub-mount, it may be possible to test the chip on the sub-mount prior to mounting on the PCB. Another advantage of using a sub-mount for a laser chip is that it may in some cases enable the adjustment of the polarisation after mounting, since the laser chip is not directly mounted on the PCB.

Where both the source and detector are mounted as COB components, this allows particularly compact opto-mechanical design, with reduced dead volumes and efficient optical coupling to any sample cell optical elements for transmission of the electromagnetic radiation into and out from the sample cell.

In some embodiments, a solid-state COB laser or other radiation source may be in direct contact with an adhesive, polymer, fluid or gel through which the output radiation is transmitted, and in this case the refractive index of this material may be specifically chosen so as not to closely match the refractive index of the solid-state laser, since this might affect the gain medium created by internal reflection at the edge of the laser structure and hence affect, reduce or even interrupt lasing action. In such a case, however, it may still be desirable to match the refractive index of the material to the optical elements at the input and/or output of the sample cell.

COB mounted solid-state sources and detectors, such as lasers and photodiodes, are vulnerable to physical, chemical and electrostatic damage through use, handling and/or compounds present in the dead volume, and so the inclusion of adhesive, polymer, fluid or gel in contact with these components may also protect the laser diode from damage, provided the material has appropriate properties, such as being able to give some physical protection, being an electrical insulator, a thermal insulator and/or being chemically inert.

In a preferred embodiment of a TDLS measurement system, the temperature of the laser diode and/or photodiode is controlled by at least one thermo-electric (or Peltier) cooler (TEC) and temperature feedback is provided by a temperature sensor, such as a thermistor, resistance temperature detector (RTD) or thermocouple. The laser temperature and current are controlled electronically to enable scanning over the desired gas absorption line. The detector output, which incorporates the photodetector and amplifiers, is used with real-time signal processing software, which may make use of direct absorption measurements or the 2^{nd} harmonic signal and its unique absorption shape characteristics, such as height and width, to determine the true gas concentration.

The use of COB components may also be optimised by using the physical design and materials of the PCB as described further below.

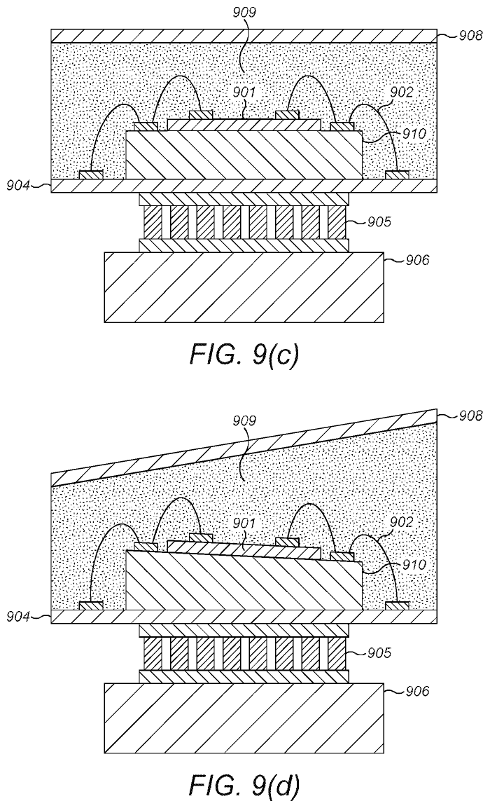

It should be noted that the components in the figures are illustrated schematically, and are not intended to represent the dimensions of the illustrated substrates, or other components, to scale. FIG. 9(a) illustrates a COB design of a laser source for use in a spectroscopic gas analysis system. In this embodiment, a laser source (901) is mounted directly on a PCB substrate (904) in a COB arrangement, and is configured to emit electromagnetic radiation into a gas detection or measurement volume, such as a gas sample cell (not shown), through an optical element (908), which may be a window, lens or mirror forming an interface with the gas detection or measurement volume. In this example, the substrate (904) is mounted on a thermo-electric cooler (TEC) (905), for controlling the temperature of the laser (901). It should be noted that the positioning of the TEC on the underside of the substrate is made possible in this configuration by the mounting of the laser source directly on the substrate as a COB component, thereby enabling close thermal coupling with the TEC. The laser source may also be mounted on the substrate using a sub-mount (not shown), which may facilitate the mounting of the laser at a selected angle, as required by the optical configuration of the laser relative to the gas detection or measurement volume, and the sub-mount may still enable close thermal coupling of the laser source with the TEC by virtue of the sub-mount comprising highly thermally conductive material.

Among the advantages of a COB design are compactness, provision of the best thermal coupling of the laser (901) to the thermo-electric cooler (TEC) (905), and the elimination of any optical fringes formed inside a conventional metal can-window enclosure of a laser diode. If both the source and detector are mounted in a COB configuration, then this may result in a particularly compact arrangement, and the components may advantageously be assembled in a one-stage process, resulting in simplified manufacturing and reduced cost.

The process of mounting a COB component consists of three main manufacturing processes. The first is the die mounting step or “die attach, which consists of applying a special thermally conductive adhesive (903) to secure the laser chip directly to the PCB substrate (904). The second is the “wire bonding” process which makes electrical connections, here in the form of wire bonds (902), between the laser chip and PCB connection pads. In some embodiments, a third process is “encapsulation”, which consists of dispensing a layer of a clear protective material over the die and the wire bonds, as shown in FIG. 9(b). This layer will conventionally be very thin. However, in accordance with embodiments described in this patent specification, the whole of the dead space void (911) between the light source and one or more optical elements such as entry and exit windows (908) of the gas cell may be filled up with appropriate optically transmissive encapsulant and/or filler material. This encapsulant/filler may negate the need for the thin protective layer, or may overlay a protective layer. This encapsulant/filler may be chosen to be approximately refractive index-matched to at least one optical element (908), which may be one or more of the following: a window, a lens, an attenuator or a band-pass filter. It should be noted that where a filler material is used to fill the dead space as shown in FIG. 9(b), the use of a COB configuration for the source and/or detector components may provide an additional advantage over the use of conventional encapsulated devices provided in metal cans, by avoiding the possibility of bubbles forming in the filler material due to gas leakage from the can.

Heat generated by the TEC (905) may be dissipated by a suitable heatsink (906) provided beneath the TEC.

Due to the small size of the laser chip and, in some embodiments, its direct thermal contact with copper areas of the PCB, a significant reduction is achieved in the amount of power required by the TEC to maintain the laser chip at a desired setpoint temperature. Additionally, the significantly lower thermal capacity of the bare chip than a conventional canned device allows for very fast and accurate thermal tuning of the laser temperature and its wavelength. To control the laser chip at a desired set-point temperature, an accompanying temperature sensor may be thermally close-coupled with the TDL. For example, a temperature sensor may be co-planar and mounted in close proximity to the TDL, and potentially co-adhered and co-encapsulated.

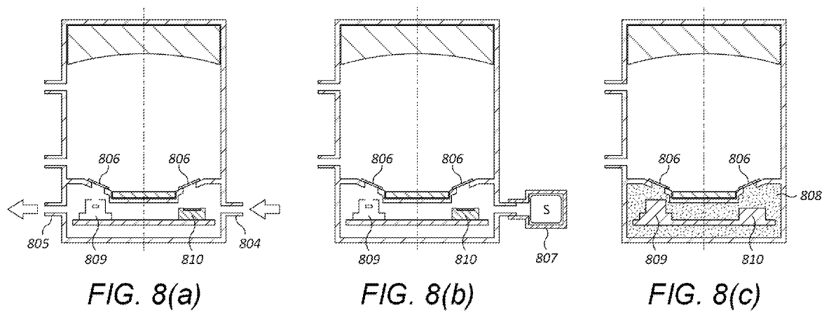

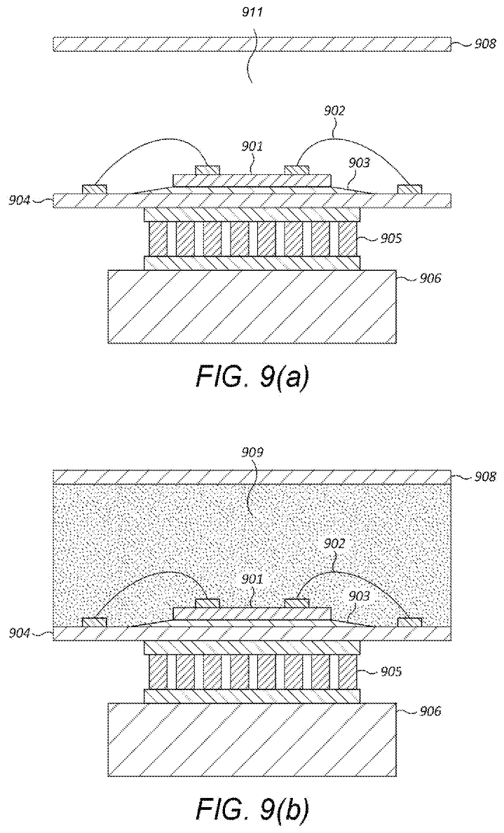

Additionally, the properties of the encapsulant or dead volume filler (909), where provided, should be approximately electrically and thermally insulative, since it is contiguous with the COB component and/or other electronic components. The above-mentioned approximate refractive index matching of the encapsulant or dead volume filler (909) to at least one optical component (908) reduces stray reflections and transmission losses, and decreases etalon formation and optical feedback into the laser. The encapsulating filler material may be chosen to specifically avoid a refractive index-match to the laser source, because this may interfere with the laser gain medium of the COB component. The transmissive encapsulating filler material (909) may be a gel, or fluid, or may include a solid insert, and may be formed of any of the filler materials described in this patent specification including polymer or adhesive materials, or another suitable filler substance. The filler material may act as a secondary physical barrier to gas ingress/egress, in case of the failure of a seal between the COB component and the sample cell. This may be particularly relevant for flammable and/or toxic gas samples. For reasons described earlier and shown in the example of FIGS. 9(c) and 9(d), there may be times when it is preferable to mount the source (laser or LED) chip and/or detector chip on a sub-mount (910). This may be for at least one of the following reasons: ease of handling, pre-testing of the chip, orientation, angle or thermal properties. This requires some extra wire bonding to mount the chip to the sub-mount as well as from the sub-mount to the PCB. As shown in FIG. 9(d), the mount may have a tapering shape (with non-parallel top and bottom surfaces) for a transmission direction that is not perpendicular to the PCB substrate (904). Adhesive layers (903) have been omitted from FIGS. 9(c) and 9(d), but adhesive layers may be used between the components shown in those figures. As shown in FIGS. 8(c) and 9(d), the windows that allow electromagnetic radiation to enter or leave the gas cell may be tilted relative to the mounted source or detector chip, for example at or close to the Brewster angle to minimise surface reflections.

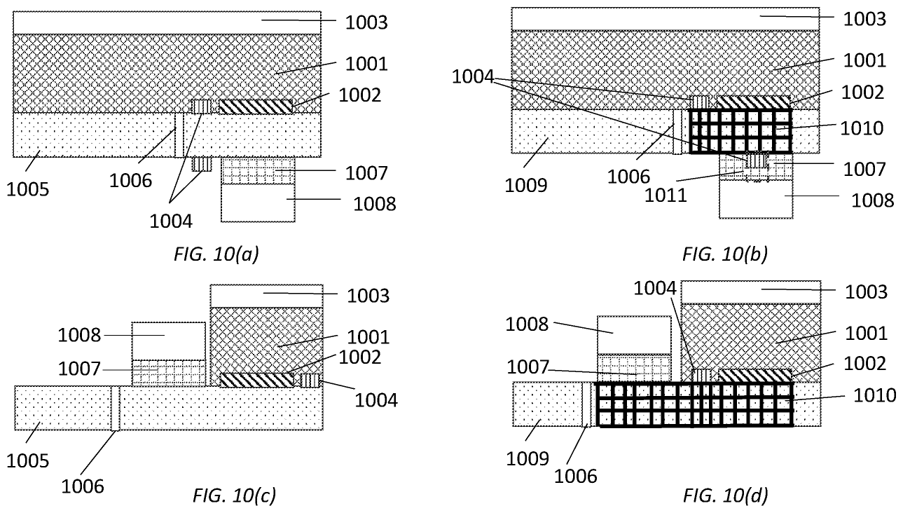

Referring to FIG. 10(a), in a preferred embodiment, at least one chip-on-board component such as a laser or LED source or detector (1002) is provided on a substrate (1005) for emitting or detecting electromagnetic radiation to or from a gas detection or measurement cell (not shown) via an optical interface (1003). As described above, a dead volume may be present between the COB component (1002) and the optical element (1003), or alternatively a gel or other suitable filler substance (1001) may be injected/poured or placed by other suitable means into the dead volume to partially or completely fill the void between the COB component and the gas cell optical elements (1003). For close temperature control, at least one temperature sensor (1004) and temperature control means (1007) may be closely thermally coupled to the COB component (1002). This close thermal coupling may be achieved, for example, by the use of a substrate (1005) comprising a highly thermally conductive and electrically insulative substrate material, such as alumina or aluminium nitride. Alternatively, a layer of highly thermally conductive metal, such as copper, or a metal alloy may be used, but may be provided with an appropriate electrically insulative layer, potentially with the use of one or more thermal breaks (1006) to provide thermal isolation between the thermally conductive material and other portions of the substrate. In the embodiment shown in FIG. 10(a), two temperature sensors (1004) are shown, located above and beneath the substrate (1005), respectively. It should be noted that the use of a COB configuration for the component (1002), which may be a laser chip, and the resulting reduction in size of the overall assembly, enables the use of two temperature sensors located on opposite sides of the substrate, due to the improved thermal coupling between the component (1002) and components located on the opposite side of the substrate. This arrangement may in turn lead to increased accuracy of temperature detection and temperature control.

The temperature control means may be a heater or thermoelectric cooler (TEC) (1007) and may be used to control the temperature of the COB component through heating and/or cooling using the Peltier effect. A heatsink (1008) may be used to dissipate any excess heat generated, and may be disposed beneath the TEC.

In an alternative embodiment shown in FIG. 10(b), a standard printed circuit board (PCB) (1009) may be used, and highly thermally conductive multi-layer vias (e.g. using gold) and inter-layer planes or conductive layers (e.g. using copper) (1010) may be provided to enhance the naturally low thermal conductivity of the typical fibre glass/resin composite structure of the PCB, whilst still avoiding undesired electrical shorting to the functional electrical circuit. In particular, as shown in FIG. 10(b), these vias and/or conductive layers may be provided at least in a region of the substrate adjacent to (e.g. directly above or beneath) the COB component (1002), temperature sensor(s) (1004) and thermal control means (1007), in order to provide improved thermal coupling between these components.

Advantageously, one or more thermal breaks (1006) may be provided between the region of the substrate which includes thermally conductive multi-layer vias and/or inter-layer planes and other portions of the substrate. In this way, the efficiency of the operation of the TEC (1007) may be increased, since the heating/cooling effect is substantially restricted to the region of the substrate where the COB component is located. This reduction in the thermal mass of the region heated and/or cooled by the TEC may also enable a smaller TEC to be used to achieve the required heating/cooling, and may enable more rapid control of the COB component, e.g. temperature control used to thermally tune the wavelength of a laser emitter. More rapid control of the emitter wavelength may enable the apparatus to be used more effectively over an increased operating range, for example in the detection of different gases in the sample cell.

The one or more thermal breaks may comprise a mechanically formed or laser-etched slot in the substrate, or any form of cutout or partial cut-through in the thickness of the substrate. For example, a slot or groove made partially through the thickness of the substrate may be provided adjacent to, or shaped to at least partially surround, one or more components, in order to provide a thermal break between that component and other components or substrate regions. Additionally or alternatively, a hole or cutout which extends through the entire thickness of the substrate may be employed for the same purpose, and may be positioned adjacent to, or partially surrounding one or more components. In this way, an air gap, or a gap filled with other material having lower thermal conductivity than the substrate material, may be provided in order to thermally isolate the region of the substrate in which the COB component is located from other regions of the substrate. An air gap, for example, provides a space between these regions which has a low thermal conductivity compared with the thermal conductivity of the substrate. In some embodiments, the thermal break may take the form of a circle, or other shape, surrounding the COB component on the surface of the substrate, in order to reduce the thermal mass of the region heated/cooled by the TEC to the immediate vicinity of the COB component and other components thermally coupled with it, as described above.

The inter-layer planes may comprise one or more layers extending substantially parallel to the surface of the PCB or other substrate, and distributed throughout the depth of the substrate so as to increase the overall thermal conductivity of the substrate, as required. When provided in combination with thermally conductive vias as shown, the vias may be arranged to act in combination with the conductive layers to transfer heat through the substrate. In particular, the vias may transfer heat from the surface of the substrate vertically to the thermally conductive inter-layer planes, which may transfer heat horizontally through the substrate. The number of vias and/or inter-layer planes may be determined based on the thermal conductivity of the substrate material and the dimensions of the substrate. For example, where a thin substrate is used, thermal coupling across the thickness of the substrate may be sufficiently good to avoid the need for the use of thermally conductive vias, but one or more planar thermally conductive layers may be employed to provide increased thermal conductivity along the plane of the substrate. It should be noted that the thermally conductive vias, where included, may also be used to provide electrical connections between PCB layers.

A thin PCB may also be used to minimise any thermal gradient within the PCB. In the case of using a heater for temperature control, this may advantageously be provided in the form of a resistive track circuit on at least one layer of the PCB structure. In this example, the heater may be conveniently incorporated into the PCB using known PCB manufacturing techniques, and may be embedded between insulating layers of the PCB to provide efficient electrical heating of a portion of the substrate, and in particular the region adjacent to the COB component for temperature control, e.g. for laser tuning. In some preferred embodiments, such as the one shown in FIG. 10(b), at least one temperature sensor (1004) is mounted directly beneath the COB component (1002), and a thermoelectric cooler (1007) positioned directly beneath the PCB (1009) is provided with an appropriate cavity or void (1011) to accommodate the temperature sensor, such that the TEC surrounds the temperature sensor. By using this arrangement, the close thermal coupling of the temperature sensor (1004) and TEC (1007) with the COB component (1002) can be optimised.

When the temperature sensor (1004) is mounted on the same side of the substrate as the COB component, it is advantageous to provide sufficient clearance between the temperature sensor and the COB component to enable surface mount positioning tools to access the mounting spaces of the various components to facilitate assembly.

In some embodiments, as shown in FIGS. 10(c) and 10(d), both the temperature sensor (1004) and the temperature control means (1007) may be co-planar with the COB component (1002). FIG. 10(c) shows an arrangement in which a COB component (1002) and a temperature sensor (1004) are mounted adjacent one another on a first surface of a substrate (1005). A temperature control means (1007), such as a thermoelectric cooler, is also mounted on the first surface of the substrate, with a heatsink (1008) disposed on the thermal control means. In the illustrated arrangement, a filler material (1001) is provided in the void between the COB component (1002) and the optical element (1003) of an adjoining gas cell, and also encloses the temperature sensor. The substrate may be provided with a thermal break (1006), as shown, to provide thermal isolation between the substrate region containing the COB component (1002), temperature sensor (1004), thermal control means (1007) and heatsink (1008), and other portions of the substrate.

FIG. 10(d) shows an arrangement similar to that of FIG. 10(c), but in which the substrate is a PCB (1009) which is provided with an arrangement of multi-layer vias and highly thermally conductive inter-layer planes (1010) to enhance the thermal conductivity of the PCB substrate material. As shown in FIG. 10(d), the arrangement of multi-layer vias and inter-layer planes may be provided at least in the region of the PCB beneath the COB component (1002), temperature sensor (1004) and thermal control means (1007), in order to provide improved thermal coupling between these components. As in the arrangement of FIG. 10(c), FIG. 10(d) shows the provision of a thermal break (1006) to thermally isolate the region of the PCB which includes the arrangement of multi-layer vias and inter-layer planes (1010) from other portions of the PCB.

The arrangements shown in FIGS. 10(c) and 10(d), in which components are located on a single surface of the substrate (1005) or PCB (1009) may be more easily manufactured than the arrangements of FIGS. 10(a) and 10(b), in which components are mounted on both sides of the substrate. However, the arrangements of FIGS. 10(a) and 10(b) may provide improved thermal efficiency compared with those arrangements in which components are disposed only on a single side of the substrate.

While FIGS. 9 and 10 show particular arrangements of the components (e.g. the COB component, temperature sensor(s), thermoelectric heater/cooler and/or heatsink) relative to the substrate, this disclosure is not limited to the specific illustrated arrangements and it will be appreciated that various modifications to the schematically illustrated arrangements are possible to achieve the described functionality.

In a preferred embodiment, when a gel or other suitable filler substance is injected or poured into the dead space, its viscosity should be chosen to allow complete flow access for avoidance of any bubble or void formation. This viscosity may be controlled by composition and/or temperature. Additionally, minimisation of (air) bubble formation may be enhanced by the use of a vacuum, when making and/or injecting/pouring the gel and/or setting the gel. Flexibility within the gel or filler structure decreases the likelihood of crack formation due to aging or temperature cycling. The presence of bubbles within the gel may decrease the performance through scattering, absorption and etalon formation.

Note that where references are made to the use of a gel as a preferred medium, this does not preclude the use of other filler material such as low, medium or high viscosity polymers, fluids or adhesives, as well as a gelatinous medium, which, when introduced into a void within the apparatus, displaces whatever gas is present and prevents its return. The properties of the polymeric gel are such that, once set, although there is still some liquid phase, it behaves somewhat like a solid due to the cross linkages between polymer molecules. The extent of cross linking and type of polymer will determine the rigidity and other features of the gel. The inventors have determined that the properties of the gel, as well as the means and environment under which it is placed within the dead volume will influence its functionality within an absorption spectrometer. For example, if the gel is injected into the dead volume cavity, the occurrence of gas (e.g. air) bubbles or voids within or around the gel may be reduced if this takes place under vacuum, but also the dead volume mechanical design, orientation with respect to gravity, temperature and gelling conditions will be taken into consideration. The viscosity of the gel whilst injecting or pouring should be chosen to allow the free movement of the gel within the dead volume cavity and to fill all of the voids and make contact with all of the exposed surfaces within the optical path of the transmitted radiation. Very small spaces and/or apertures within the dead volume should be avoided whenever the apparatus design constraints allow this, as well as very high surface roughness being avoided where possible, since this may inhibit free ingress of the gel. The speed at which the gel solidifies may be influenced by composition, catalytic action and temperature.

The gel may be chosen to be transparent or almost transparent to the wavelength range of interest or, conversely, the gel may be selected to attenuate the radiative output from the source. Attenuation may be desirable, for example, if the optical intensity of the unattenuated radiation would saturate the detector or if the intensity could potentially cause ignition within a flammable mixture. This attenuation could be due to the intrinsic properties of the gel and wavelength used or the material could be doped with materials and/or dyes to absorb some of the radiative throughput. The gel may also act as a secondary barrier for leaks to and from the sample cell. This could be especially relevant for flammable and/or toxic samples. The gel should preferably have low permeability to relevant and/or interfering gases and be mechanically stable over time, i.e. should not shrink back or change spectroscopic properties.

Although the gel's primary function is to fill the dead volume and eliminate gaseous optical absorption, if the refractive index of the gel is closely matched to that of one or more of the optical elements of the absorption spectrometer (source, detector, gas cell optical inlet/outlet), it may reduce back reflection/increase transmission and hence enhance optical throughput and attenuate optical interference and optical feedback into a solid-state laser (if used). Although refractive index matching gels are already used in various optical systems, such as compound lenses and fibre optic coupling to reduce interface effects between individual optical components, that implementation and purpose is very different from that being described within this patent specification. In some instances, where there is significant refractive index variation between optical components, a graded refractive index gel may be desirable to minimise reflective losses. This may be achieved by layering different gel compositions and/or through doping the material or through other suitable means.

It may be desirable to have at least one fluid (gas or liquid) reference volume encapsulated within the gel structure of the dead volume (fluid reference bubble). This may be used as a line-lock reference, potentially containing the gas of interest or other suitable gas within the wavelength measurement range, optical absorption interference reduction or other appropriate function. This may be achieved by injecting a known fluid into the gel using a syringe under controlled conditions (such as location, composition, temperature and pressure of the fluid) or by other suitable means. Note that the location of the gel with reference bubble may be located close to the line-lock detector for TDL measurements, potentially eliminating the need for other suitable reference means, such as a sealed reference cuvette.

If electrical or magnetic fields are deployed across the gel, the physical and spectroscopic properties of gel in certain preferred embodiments should be only minimally or completely unaffected by the presence of these fields, so as to minimally affect the spectroscopic measurement.

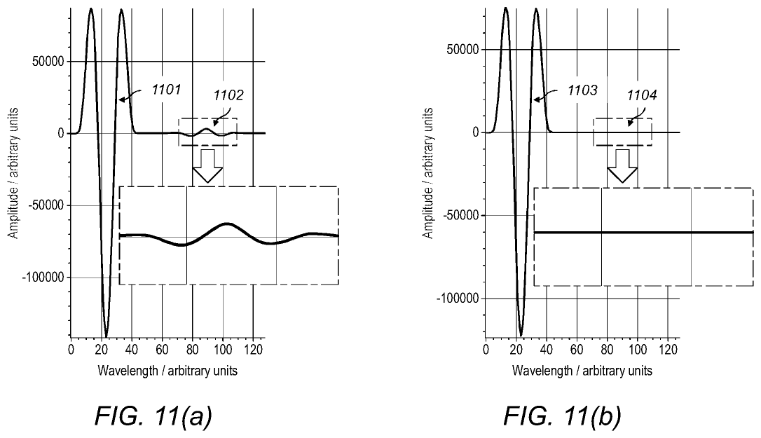

FIG. 11 illustrates the beneficial effect of injecting an example commercially available silicone-based gel (Avantor NuSil LS1-3252) into the physical apparatus shown in FIG. 8(c), with a non-absorbing gas (nitrogen) flowing through the sample cell. In this illustration, the target gas (oxygen) is detected by scanning the electromagnetic radiative output from a Chip on Board VCSEL across an oxygen absorption line, by tuning the laser drive current and analysing the signal using WMS (wavelength modulation spectroscopy). FIG. 11(a) shows the laser intensity reference signal 1101, and the absorption signal due to residual oxygen present in the dead volume (when no gas-displacing filler material is present) is also shown by the arrow 1102. FIG. 11(b) shows the laser intensity reference signal 1103 and the absorption signal due to residual oxygen present in the dead volume 1104 after the optically transmissive filler gel has been injected into the cavity. It can clearly be seen that the gel has allowed transmission of electromagnetic radiation through the dead volume at this wavelength range with less absorption than in FIG. 11(a). This is because the gel has displaced oxygen from the dead volume. For ease of comparison, both FIGS. 11(a) and 11(b) show zoomed-in features of the oxygen absorption zone.

Other features and functions may also be integrated in and around the cell in order to enhance its optical performance, environmental stability and signal processing. These optional features and functions may include optical elements including windows, lenses, mirrors, attenuators, optical band pass filters, or reference cells for line locking and/or validation. Additionally, physical and spectroscopic features can be used to minimise stray optical reflections, such as using surface roughening and/or blackening, or environmental factors including gas flow, temperature and pressure may be controlled and/or corrected. In some examples, flow and/or diffusional features (such as gas ports, diffusional exchange membranes) and volume reduction features are used to reduce response time. In some examples, the application of electrical and/or magnetic fields to the measurement cell and/or other regions, and/or path modulation is used to reduce etalon effects.

Signal processing, including frequency domain, filtering and averaging techniques may also advantageously be employed.

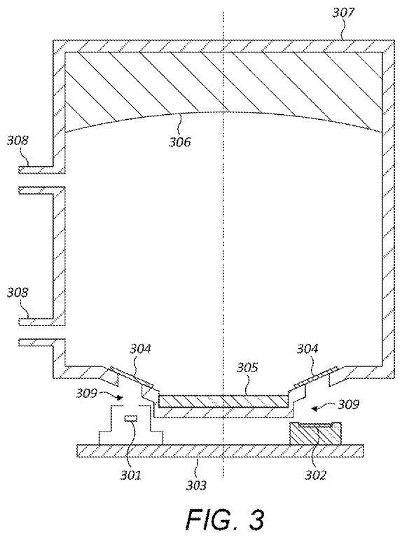

As mentioned above, the invention may be implemented, for example, in an absorption spectroscopy apparatus including a folded-path gas sample cell, as described in UK Patent Application No. 2304895.2. As illustrated in the example of FIG. 3, a compact absorption spectroscopy apparatus may include at least one source (301) of electromagnetic radiation is present for transmitting electromagnetic radiation for transmitting electromagnetic radiation through a gas sample contained within the gas cell (307) and towards at least one detector (302). The gas cell (307) for containing a gas sample or calibration gas has at least one gas exchange port (308), at least one optical element (304) for allowing transmission of electromagnetic radiation of a desired wavelength range in and out of the gas cell, and two or more mirrors (305, 306) arranged in opposed relation to each other, including at least one curved mirror (306) arranged to reflect the transmitted electromagnetic radiation towards a second mirror (305), wherein the second mirror (305) is arranged to reflect the electromagnetic radiation back towards the at least one curved mirror, and wherein said two or more mirrors (305, 306) are arranged to reflect the electromagnetic radiation in a folded optical path through the gas sample towards the at least one detector (302) such that a transmitted beam of electromagnetic radiation is directed towards the at least one detector (302). This arrangement using one or more curved mirrors can provide automatic focussing of a transmitted diverging beam such that it converges towards the detector after being reflected within the gas cell. However, the apparatus can also be used with a laser source whose collimated beam is reflected towards the detector; the curved mirrors provide a degree of tolerance to manufacturing variations both for diverging and collimated beams. The at least one detector (302) monitors absorption of electromagnetic radiation for at least one absorption wavelength or wavelength range associated with at least one gas species, by detecting transmitted electromagnetic radiation that is not absorbed and at least one analyser is used for analysing an output signal from the at least one detector to determine the presence and/or measure a parameter of at least one gas species within the gas sample.

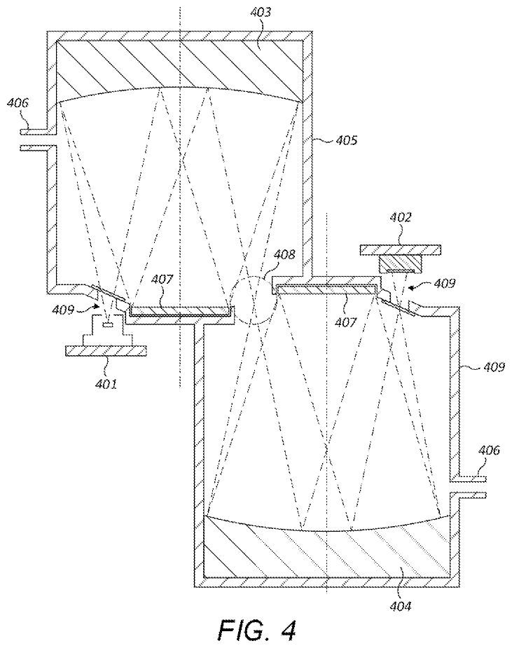

FIG. 4 illustrates a modular implementation of the device from FIG. 3. In the embodiment of FIG. 4, radiation from the source (401) enters the sample cell (405) and is reflected by a first curved or spherical mirror (403) onto a second mirror (407), back onto the first mirror (403), which then focuses the light as it exits the first cell (408) which then becomes the input into the second cell (409). In a likewise manner, the light is reflected by the mirrors (404) and (407) within the cell (409) and is focused onto the detector (402). Sample gas is allowed to pass through the cell via inlet and outlet ports (406).

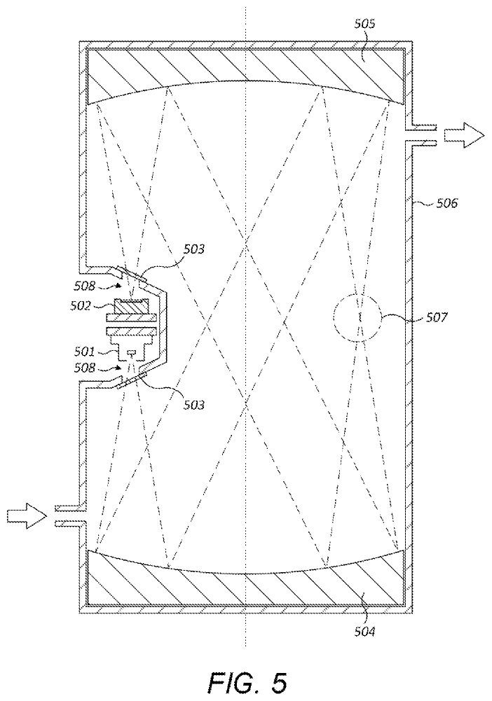

Another preferred embodiment from UK Patent Application No. 2304895.2 is illustrated in FIG. 5, for an embodiment with a longer pathlength per cell length. This embodiment achieves an enhanced pathlength with simplicity of design and build compared to a modular system of two of the earlier-described embodiments (FIG. 4). The diverging beam from the source (501) passes through the optical element (503) and enters the sample cell (506). Inside the sample cell multiple reflections occur between the first mirror (504) and a second mirror (505) as illustrated in FIG. 5. In such a simplified embodiment, mirrors (504) and (505) may be identical cross-sectional slices of a spherical section. The outgoing beam passes through the optical element (503) and is incident on the detector (502). A wavelength lock cell may be included, which may be preferably located at position (507), where a localised focal point forms and an optical element may be used to divert a small fraction of the beam through an optionally placed wavelength lock cell.

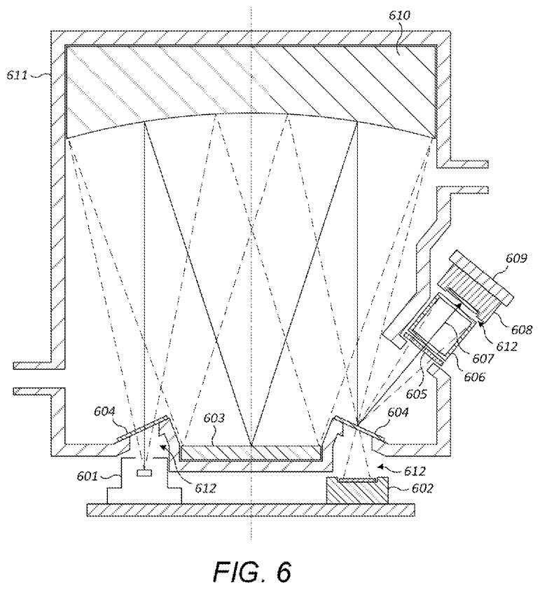

The stability of the scan current and the laser diode characteristics are paramount to the wavelength stability of the TDLS measurement. It is vital that in the case of either electronics or laser diode drift over time, the measurement can not only remain “locked” to the desired absorption line, but also produce a diagnostic report about the extent of this drift as a measure of preventative maintenance. A simple approach would be to enable an algorithm that detects the location of the gas absorption 2^{nd} harmonic peak, and actively maintains this location in a feedback loop, by continuously fine adjusting the set point temperature of the laser diode. However, this approach assumes that the process gas flowing through the cell always contains some desired gas, which produces a measurable 2^{nd} harmonic peak. In practice, no such assumption can be made. Real life processes may be highly variable and, for long periods of time, may contain either no desired gas, or, even worse, may contain a gas with a neighbouring interfering absorption line. For this reason, the instrument is ideally fitted with an on-board “wavelength reference” device, which, in a preferred embodiment, constantly monitors a real spectroscopic 2^{nd} harmonic signal from a known reference gas, which is always present regardless of the process variations. Ideally, this reference gas can be the main gas of interest or another surrogate gas with absorption lines nearby. For practical cost and size constraints, this reference device should be as compact and low cost as possible. FIG. 6 illustrates one of the preferred embodiments of a TDLS instrument, where the diverging beam from the source (601) passes through the optical element (604) and is then reflected by the first (610) and second mirrors (603) and, after passing the second optical element (604), is focused on the detector (602). This detector (602) is used for the primary measurand detection. In a preferred embodiment, a small proportion of the light incident on the optical element (604) in front of the detector (602) is reflected through a low volume sealed gas capsule (cuvette) (606), with transmissive optical elements (605) to the relevant wavelength range and filled with up to 100% concentration of the reference gas (607) onto a secondary detector (608) mounted on a printed circuit board (609) for line lock to be maintained. It is important that sufficient optical absorbance of the reference gas in the reference cell takes place to create a detectable 2^{nd} harmonic signal. This is achieved by a combination of sufficient pathlength and gas fill pressure.

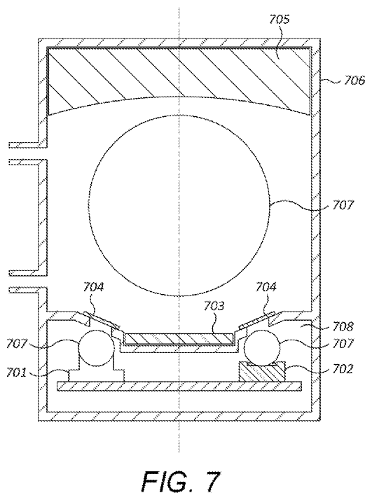

As previously described, magnetic and/or electric fields (FIG. 7) may be applied to any external volume or zone of the apparatus that is outside the detection/measurement volume but within the optical path, and/or may be applied to the gas detection/measurement volume. The diverging beam from the source (701) passes through the optical element (704) into the sample cell (706), where it is reflected by the first (705) mirror and the second mirror (703) and focused through a second optical element (704) onto the detector (702). Magnetic and/or electrical fields may be applied (707) over the external volumes (or “dead zones”) and/or over the sample cell regions. The presence of a magnetic and/or electric field may induce splitting of the absorption lines by the Zeeman and/or Stark effects, which may be used advantageously for enhanced signal processing.

FIG. 8 illustrates the void or dead space between the at least one source (809) and/or at least one detector (810) and the at least one optical element (806) on the sample cell. This may be a cause of measurement uncertainty, due to the presence of the gas species of interest or an optical interferent. This interfering gas species may be present from the time of manufacture and/or through diffusion from the surrounding environment, and could be variable. This may be mitigated by several traditional means, such as shown in FIGS. 8(a) and 8(b). Known solutions include flushing of the dead space with a non-optically absorbing gas, such as nitrogen (FIG. 8(a)) and/or chemical scrubbing for a specific gas or specific gases (FIG. 8(b)). With reference to FIG. 8, a more detailed explanation of the above options will be given. In FIG. 8(a), the dead space is sealed by the optical elements (806). However, it is purged with a non-optically interfering gas. This has the advantages of eliminating the measurement uncertainty, but has the disadvantage of needing a continuous supply of purge gas with increased complexity and cost. In FIG. 8(b), where a scrubber (807) is used to eliminate the interfering gases in the dead space, this has the advantage of not requiring a continuous supply of purge gas. However, this requires a high integrity seal and the scrubber may become saturated over time requiring replacement. The scrubber material should be selected according to the application, increasing complexity and cost. Alternatively, as shown in FIG. 8(c), a dead space (808) (or any secondary volume that is within the optical path but separated from the detection and/or measurement volume) may be advantageously filled with a gel or other suitable filler substance such as a fluid, polymer, insert, adhesive or combination of filler substances, which is at least partially transmissive to the wavelength range of interest and substantially impermeable to interferent gases that might otherwise absorb photons having certain energies (corresponding to particular wavelengths of the transmitted electromagnetic radiation) and thereby reduce the accuracy of measurements of absorption. The gas-impermeable and transmissive material may be refractive index matched to the optical element materials of the source (809) and/or the detector (810), and/or matched to the optical elements (806) of the cell. As noted above, absorption spectroscopy is known for use in gas analysis, including for determining the presence of at least one particular gas species in a measurement volume and for measurement of parameters including concentration of the individual gas species in a gas sample. The use of reflective optics has advantages over refractive optics for several reasons including a having a higher optical throughput (greater transmission) and being independent of wavelength (chromatic aberration), which is especially important for a modular system for the detection of many different gas species at different wavelengths. However, refractive optics may still be usefully employed in many embodiments.

Multi-gas measurements may also take place, where two or more lasers and/or detectors are used and the wavelengths of the sources are chosen to correspond to the absorption wavelengths of interest for at least two different measurands. Likewise, the detectors are chosen to have responsivities in the wavelengths of interest for the measurands. In some applications more than two matched pairs of sources and detectors may be used depending on the measurands and the sizes of the components and the optics used.

In some preferred embodiments, signal processing may take place using analogue and/or digital electronics, including the use of multiplexed ADCs and/or processors.

Any reflective surfaces used may be made of machined and/or moulded metal, glass or polymer and polished as required and may have reflective and/or protective coatings such as gold. Any refractive optical elements used may be made of machined or moulded polymer or glass and polished as required and may have optically protective and/or anti-reflective and/or absorptive coatings. It may become important, especially for coherent sources, such as lasers, to suppress stray reflections from being collected at the detector, since constructive and destructive interference effects (etalons) could occur. The suppression of these stray reflections may take the form of random surface roughening to disrupt specular reflection and/or blackening (optically absorptive coating) of the relevant surfaces. Any such applied optically absorptive coating or material should ideally be chemically compatible with the gas sample that it is in contact with and approximately a perfect light absorber at the optical wavelength range being used. Alternatively, in some preferred embodiments, for example, with broadband (e.g. for non-coherent LED or incandescent) sources, stray reflections which arrive at the detector may actually be useful, to increase the overall optical throughput and give improved signal to noise and therefore no suppression of reflections is needed and/or these stray reflections may even be enhanced, for example, by the application of a reflective layer to at least one surface within the sample cell, such as a gold layer.

Any interaction with at least one optical element or reflective feature such as an attenuator, pass band filter, window, lens, polariser (e.g. for use if not all the laser output is intrinsically polarised) or reflective surface may cause an etalon to form. In general, the number of optical elements should be kept to a minimum, to minimise etalon formation, but there will always be some elements or features present, such as windows for the source and/or detector and for light entering into and exiting the sample cell. In some applications, where the stray light reflections should be minimised as discussed in the previous section, the formation of such etalons may be minimised by using anti-reflective coatings and/or angled and/or wedge windows. In addition, dimensional changes may cause shifts in the etalons and hence affect the signal. This can be reduced by the use of low thermal expansion coefficient materials such as invar and/or using a temperature-controlled cell. The use of a temperature-controlled cell and/or pre-equilibrator for the sample gas, so the incoming gas is in equilibrium with the cell temperature will increase signal stability.

The magnitude of the etalons and changes with temperature may be reduced by the use of at least one pathlength modulator. The at least one modulator may be employed at different locations within the system, depending, amongst other considerations, on the locations of the most relevant etalon producing features and the overall opto-mechanical arrangement. The modulator may be a solid-state device, such as a piezo-electric device, whose dimensions may be changed via the application of a voltage, although consideration of the voltage magnitude should be taken into account for potentially flammable mixtures. Alternatively, the modulator may be an electromechanical device, such as a voice coil type arrangement in combination with a permanent magnet, similar in design to a loudspeaker or an electromechanical vibrating element. A mechanical flexure may be employed, such as a spring or elastic polymer or foam, to act as a director and damping element. In the case of a foam being used, a sealed cell format may be preferentially employed to modify the physical properties of the flexure such as density and/or elastic properties without entraining sample gas. In some embodiments, modulations on pathlength may be preferentially applied to the approximately spherical mirror slice cross-section (306), since this has less influence on the focussing position at the detector than if the approximately flat or concave mirror (305) were modulated. The frequency of any such modulation should take into account the functional scan speed in the case of a diode laser measurement or pulse rate in the case of LED's or pulsed incandescent sources and the expected response time, so as not to detrimentally influence the measurement. The amplitude of the modulation should ideally be greater than the wavelength of light being used, however the frequency and amplitude may be chosen theoretically and/or empirically to give the desired performance and/or lifetime of the modulating element.

When designing the opto-mechanics of the sample cell and related optics there are two important considerations to take into account:
It should provide optimum path length for the specific gas range, whilst, ideally, having a small sample flush volume for a fast response time.
It should produce a low level of optical fringes, when used with a diverging coherent source such as a laser diode.

It is envisaged by the inventors that there are potentially many different embodiments using the design principles described in this patent to form an absorption spectrometer having a source and/or detector in a COB format, and optionally including a gel-filled dead space.

In some embodiments, the source and/or detector is remote-mounted and the electromagnetic radiation is conducted to and from the apparatus using at least one fibre optic cable. This may have advantages where temperature and/or electromagnetic interference might render direct coupling impractical.

What is claimed is:

1. An apparatus for use in absorption spectroscopy, comprising:
at least one source of electromagnetic radiation for transmitting electromagnetic radiation along an optical path that passes through a gas measurement volume, towards at least one detector;
at least one detector to detect the transmitted electromagnetic radiation after passing through the gas measurement volume and to provide an output signal indicative of the detected electromagnetic radiation; and
an analyser connected to the at least one detector to receive the output signal and analyse the effects of absorption by at least one gas species within the gas measurement volume for at least one wavelength range of the transmitted electromagnetic radiation, thereby to detect or measure a parameter of the at least one gas species;
wherein at least one source or detector comprises a Chip-on-Board (COB) component comprising a solid-state source and/or detector of electromagnetic radiation mounted onto a substrate in a COB configuration.

2. The apparatus according to claim 1, wherein the substrate is thermally conductive.

3. The apparatus according to claim 2, wherein the substrate is electrically insulative.

4. The apparatus according to claim 2, wherein the substrate comprises alumina or aluminium nitride.

5. The apparatus according to claim 2, wherein the substrate comprises a multi-layered printed circuit board (PCB) including thermally conductive vias and/or one or more thermally conductive layers for transferring heat through the substrate.

6. The apparatus according to claim 2, wherein the substrate comprises at least one thermal break for providing thermal isolation of at least one COB component from other regions of the substrate.

7. The apparatus according to claim 6, wherein the thermal break comprises a partial cut-through in the thickness of the substrate, at least partially surrounding the at least one COB component.

8. The apparatus according to claim 6, wherein the thermal break comprises one or more holes or cutouts extending through the thickness of the substrate and arranged adjacent to, or partially surrounding, the at least one COB component.

9. The apparatus according to claim 1, wherein at least one COB component is mounted onto the substrate via a sub-mount, for positioning the COB component at a selected spacing and/or angle relative to the substrate.

10. The apparatus according to claim 9, wherein the COB component is a detector of electromagnetic radiation and the spacing and/or angle are selected to reduce optical back-reflections from the detector.

11. The apparatus according to claim 9, wherein the sub-mount is thermally conductive.

12. The apparatus according to claim 9, wherein the sub-mount is electrically insulative.

13. The apparatus according to claim 12, wherein the sub-mount comprises alumina or aluminium nitride, or an electrically conductive metal, metal alloy or composite structure with an electrically insulative layer.

14. The apparatus according to claim 1, further comprising at least one temperature sensor located proximate to, and thermally coupled with, at least one COB component and arranged to measure the temperature of the at least one COB component.

15. The apparatus according to claim 14, wherein at least two temperature sensors are located on opposite sides of the substrate.

16. The apparatus according to claim 14, wherein the temperature sensor is arranged as part of a feedback system to control the temperature of the at least one COB component.

17. The apparatus according to claim 16, further comprising at least one heater or thermoelectric cooler configured to control the temperature of at least one COB component.

18. The apparatus according to claim 17, wherein at least one heater or thermoelectric cooler is located on the opposite side of the substrate from the at least one COB component.

19. The apparatus according to claim 18, wherein a thermoelectric cooler is located on the opposite side of the substrate from the at least one COB component, and the feedback system comprises a temperature sensor disposed on the same surface of the substrate as, and surrounded by, the thermoelectric cooler.

20. The apparatus according to claim 17, wherein at least two of the temperature sensor, COB component, heater or thermoelectric cooler are disposed on opposite sides of the substrate.

21. The apparatus according to claim 17, wherein at least one heater or thermoelectric cooler is located adjacent to the at least one COB component on the same side of the substrate, in a thermally conductive region of the substrate.

22. The apparatus according to claim 17, further comprising a heat sink disposed in contact with a thermoelectric cooler.

23. The apparatus according to claim 1, wherein the source comprises a laser chip, the substrate comprises a PCB, and the laser chip is mounted in a COB configuration in direct contact with a copper region of the PCB.

24. The apparatus according to claim 23, wherein the PCB comprises a resistive track configured as a heating circuit to control the temperature of the laser chip.

25. The apparatus according to claim 1, wherein at least one COB component is mounted to the substrate using a thermally conductive adhesive.

26. The apparatus according to claim 1, wherein at least one detector comprises a solid-state photodiode infrared detector based on a silicon, InSb, InGaAs, InAsP, InAlGaAs, InAsSb, PbS or PbSe crystal structure.

27. The apparatus according to claim 1, wherein at least one source comprises a vertical-cavity surface-emitting laser (VCSEL), distributed feedback (DFB) laser or discrete mode (DM) laser.

28. The apparatus according to claim 27, wherein the laser is an infrared laser based on an InP, GaAs, InGaAs, InAlGaAs or InAsSb crystal structure.

29. The apparatus according to claim 1, wherein the at least one source and at least one detector are arranged in positions relative to the gas measurement volume such that there is at least one void in the optical path, between the source and the gas measurement volume and/or between the detector and the gas measurement volume, wherein the at least one void is filled with an optically transmissive filler material.

30. A method of constructing an apparatus for use in absorption spectroscopy, comprising the steps of:
providing at least one source of electromagnetic radiation, for transmitting electromagnetic radiation along an optical path that passes through a gas measurement volume, towards at least one detector; and
providing at least one detector to detect the transmitted electromagnetic radiation after passing through the gas measurement volume and to provide an output signal indicative of the detected electromagnetic radiation;
wherein at least one source or detector comprises a solid-state source and/or detector of electromagnetic radiation, and the method further comprises:
mounting the solid-state source and/or detector onto a substrate in a Chip-on-Board (COB) configuration to form a COB component;
wire-bonding the COB component to form electrical connections between the COB component and connection pads provided on the substrate; and
encapsulating the COB component with a layer of protective material.

31. The method according to claim 30, wherein the substrate comprises a multi-layered printed circuit board (PCB) including thermally conductive vias and/or one or more thermally conductive layers for transferring heat through the substrate.

32. The method according to claim 31, wherein the COB component is mounted in a thermally conductive region of the substrate, and the method further comprises forming a partial cut-through in the thickness of the substrate, at least partially surrounding the COB component to provide a thermal break for providing thermal isolation of the COB component from other regions of the substrate.

33. The method according to claim 30, wherein the COB component is mounted in a thermally conductive region of the substrate, and the method further comprises forming one or more holes or cutouts extending through the thickness of the substrate and arranged adjacent to, or partially surrounding, the at least one COB component to provide a thermal break for providing thermal isolation of the COB component from other regions of the substrate.

34. The method according to claim 30, wherein the COB component is disposed on a sub-mount, and the mounting step comprises mounting the sub-mount onto the substrate in a COB configuration for positioning the COB component at a selected spacing and/or angle relative to the substrate.

35. The method according to claim 30, wherein the at least one source and at least one detector are arranged in positions relative to the gas measurement volume such that there is at least one void in the optical path, between the source and the gas measurement volume and/or between the detector and the gas measurement volume, and the encapsulating step comprises flowing an optically transmissive filler material into the at least one void to substantially fill the void with the optically transmissive filler material.

36. The method according to claim 35, wherein the encapsulating step comprises applying the layer of protective material over the COB component, and further flowing a separate optically transmissive filler material into the at least one void to substantially fill the void with the optically transmissive filler material.
